(19) United States
(12) Patent Application Publication
(10) Pub. No.: US 2026/0109670 A1
(43) Pub. Date: Apr. 23, 2026

(54) METHOD FOR ARYL DEBROMINATION AND ACYLATION OF BROMINATED BENZO-FUSED NITROGEN HETEROCYCLE

(71) Applicant: Shanghai OuChem Bio-Technology Co., LTD, Shanghai (CN)

(72) Inventors: Shaofang ZHOU, Shanghai (CN); Congrui WU, Shanghai (CN); Shuyang Chen, Shanghai (CN); Yonglei Zhou, Shanghai (CN); Fang Ling, Shanghai (CN)

(73) Assignee: Shanghai OuChem Bio-Technology Co., LTD, Shanghai (CN)

(21) Appl. No.: 19/166,498

(22) PCT Filed: Nov. 21, 2024
(86) PCT No.: PCT/CN2024/133438

§ 371 Date: Sep. 18, 2025

(30) Foreign Application Priority Data
Aug. 26, 2024 (CN) 202411176905.4

(51) Int. Cl. C07D 209/08; C07D 209/30; C07D 209/34; C07D 209/86; C07D 235/06; C07F 7/18

(52) U.S. Cl. CPC C07D 209/08; C07D 209/30; C07D 209/34; C07D 209/86; C07D 235/06; C07F 7/1892

(57) ABSTRACT

A method for aryl debromination and acylation of a brominated benzo-fused nitrogen heterocycle includes: mixing a solution including the brominated benzo-fused nitrogen heterocycle with a solution including a dehydrogenation reagent, and conducting a first reaction to produce a first material; mixing the first material with a solution including a debromination reagent, and conducting a second reaction to produce a second material; and allowing the second material to undergo a third reaction with a solution including an amide compound, such that a nucleophilic substitution reaction occurs at a substitution position of original bromine on aryl of the brominated benzo-fused nitrogen heterocycle to produce an acylated substituent; and after the reaction is completed, conducting quenching and a post-treatment to produce an acyl-substituted benzo-fused nitrogen heterocycle. The brominated benzo-fused nitrogen heterocycle is selected from one of a brominated indole, a brominated benzimidazole, a brominated indazole, a brominated indoline, and a brominated carbazole.

### TECHNICAL FIELD

The present disclosure relates to the field of compound synthesis, and specifically to a method for aryl debromination and acylation of a brominated benzo-fused nitrogen heterocycle.

### BACKGROUND TECHNOLOGY

Benzo-fused nitrogen heterocycles are important fine chemical raw materials widely present in nature. Due to unique chemical structures and biological activities, benzo-fused nitrogen heterocycles are extensively used in industries such as pesticides, pharmaceuticals, fragrances, dyes, feeds, foods, and additives. Thus, the synthesis of benzo-fused nitrogen heterocycles has long attracted widespread attention and research. Among various synthetic methods for benzo-fused nitrogen heterocycles, the direct functional group transformation is one of the most efficient methods. Acyl is a highly reactive transforming moiety that can be easily converted into other functional groups. Consequently, acyl-substituted benzo-fused nitrogen heterocycles are frequently adopted as starting materials for synthesizing pharmaceutical intermediates and active pharmaceutical ingredients. For example, indole-5-carbaldehyde produced by substituting the C5 position of aryl in indole with formyl is an important raw material. Indole-5-carbaldehyde can be used in the preparation of Naratriptan for treating migraine headaches. Indole-5-carbaldehyde can also be used to prepare 5-piperazinyl methyl-NI-aryl sulfonyl indoles, which can act as 5-hydroxytryptamine 6 receptor (5-HT6R) ligands and are potential prodrugs for treating schizophrenia and Alzheimer's disease [ACS Med. Chem. Lett. 2010, 1, 340-344]. Therefore, it is of great significance to develop processes for industrial synthesis of formyl-substituted benzo-fused nitrogen heterocycles.

Currently, the major method for preparing acyl-substituted benzo-fused nitrogen heterocycles is the following two-step and one-pot process: sodium amide, a strong alkali, is subjected to dehydrogenation with the corresponding bromide compound under reflux with heating to produce a sodium salt intermediate, and the sodium salt intermediate is subjected to debromination with tert-butyllithium and then to a nucleophilic reaction with a nucleophile to ultimately produce an aryl-substituted indole derivative [J. Org. Chem. 1986, 51 (26): 5106-5110]. This method requires harsh reaction conditions, and needs to be implemented at −78° C. in the absence of water and oxygen, resulting in difficult large-scale production.

In addition to brominated benzo-fused nitrogen heterocycles, boronic acid-substituted N-Boc benzo-fused nitrogen heterocycles can be subjected to a reaction at room temperature for 36 h with tetrahydroquinoline as a catalyst and glyoxylic acid as an acylation agent to produce N-Boc-substituted aryl-substituted indole derivatives. This method requires mild conditions, but involves a long reaction time, a low reaction yield, and the pre-synthesis of a reaction raw material. Additionally, the deprotection is required for a product to produce the final indole-5-carbaldehyde product, which increases the difficulties in reaction and post-treatment processes and leads to a relatively-low yield [Angew. Chem. 2017, 129, 8313-8317].

The above methods demonstrate disadvantages such as harsh reaction conditions, cumbersome procedures with a high production cost, or pre-treatment/post-treatment for both a reaction raw material and product and a low yield. Consequently, using these methods it is difficult to achieve a large-scale industrial production.

### CONTENT OF THE INVENTION

In view of the above technical defects, the present disclosure provides a method for aryl debromination and acylation of a brominated benzo-fused nitrogen heterocycle. The method of the present disclosure involves simple operations, readily-available raw materials, a high yield, and an easy post-treatment, is suitable for large-scale industrial production, and enables the efficient synthesis of acyl-substituted benzo-fused nitrogen heterocycles.

To achieve the above objective, the present disclosure is achieved through the following technical solutions:
A method for aryl debromination and acylation of a brominated benzo-fused nitrogen heterocycle is provided, including the following steps:
S1, mixing a solution including the brominated benzo-fused nitrogen heterocycle with a solution including a dehydrogenation reagent, and conducting a first reaction to remove active hydrogen from a nitrogen heterocyclic ring to produce a first material;
S2, mixing the first material with a solution including a debromination reagent, and conducting a second reaction to produce a second material; and
S3, allowing the second material to undergo a third reaction with a solution including an amide compound, such that a nucleophilic substitution reaction occurs at a substitution position of original bromine on aryl of the brominated benzo-fused nitrogen heterocycle to produce an acylated substituent; and after the reaction is completed, quenching the reaction, and conducting a post-treatment to produce an acyl-substituted benzo-fused nitrogen heterocycle,
where the brominated benzo-fused nitrogen heterocycle is selected from one of a brominated indole, a brominated benzimidazole, a brominated indazole, a brominated indoline, and a brominated carbazole;
the dehydrogenation reagent is aminoalkylsilane; and the debromination reagent is C₁-C₂₀ n- or iso-alkyllithium.

Further, the brominated benzo-fused nitrogen heterocycle has one of the following chemical structures:

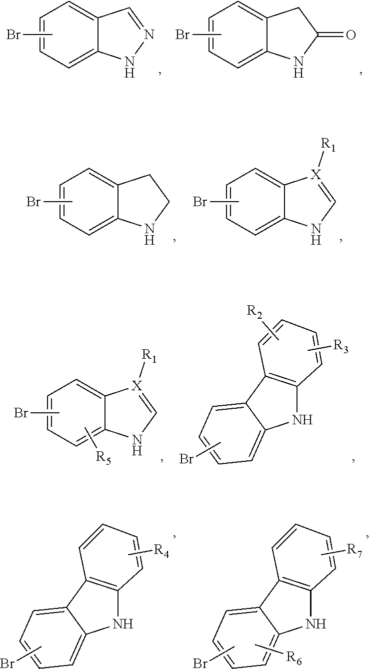

where X is selected from a nitrogen atom or a carbon atom, where when X is the nitrogen atom, there is no R₁, and when X is the carbon atom, there is R₁ selected from one of a hydrogen atom, C₁-C₆ n- or iso-alkyl, and a chlorine atom; R₅ is selected from halogen atoms, such as a fluroine atom;
R₂ and R₃ are each selected from one of a hydrogen atom, C₁-C₆ n- or iso-alkoxy, and C₁-C₆ n- or iso-alkyl, and R₂ and R₃ are the same or different;
R₄ is selected from one of a hydrogen atom and C₁-C₆ n- or iso-alkoxy;
R₆ and R₇ are each selected from one of a hydrogen atom and C₁-C₆ n- or iso-alkyl, and R₆ and R₇ are the same or different;
the aminoalkylsilane is selected from sodium bis(trimethylsilyl)amide, potassium bis(trimethylsilyl)amide, and lithium bis(trimethylsilyl)amide;
the alkyllithium is n-butyllithium; and
the amide compound is selected from one of N,N-dimethylformamide (DMF), N,N-dimethylacetamide, N-methoxy-N-methyl-benzamide, N-methoxy-N-methyl-acetamide, and N-methyl-N-methoxy-[(2S)—O-(tert-butyldimethyl silyl)-N′-(ethoxyformyl)]propanamide.

Still further, the brominated benzo-fused nitrogen heterocycle is selected from one of 4-bromoindole, 5-bromoindole, 6-bromoindole, 7-bromoindole, 6-fluoro-5-bromoindole, 7-fluoro-6-bromoindole, 3-methyl-7-bromoindole, 3-ethyl-7-bromoindole, 3-chloro-7-bromoindole, 3-chloro-5-bromoindole, 3-bromocarbazole, 8-methoxy-3-bromocarbazole, 1-methyl-3-bromo-carbazole, 1,7-dimethyl-3-bromo-carbazole, 1,6-dimethyl-3-bromo-carbazole, 7-methoxy-3-bromo-carbazole, 3-bromo-6-methoxy-carbazole, 6,7-dimethoxy-3-bromo-carbazole, 4-bromobenzimidazole, 5-bromobenzimidazole, 7-bromo-indazole, 5-bromo-indazole, 5-bromo-indoline, and 5-bromo-2-oxoindoline.

Further, the first reaction is conducted in a first continuous flow microchannel reactor, the second reaction is conducted in a second continuous flow microchannel reactor, and the third reaction is conducted in a third continuous flow microchannel reactor; the first continuous flow microchannel reactor, the second continuous flow microchannel reactor, and the third continuous flow microchannel reactor communicate with each other through microchannels; and a respective raw material is injected into a reactor for each reaction through a syringe pump.

Still further, the first continuous flow microchannel reactor includes three glass chips, the second continuous flow microchannel reactor includes five glass chips, and the third continuous flow microchannel reactor includes two glass chips; and a liquid-holding volume of each glass chip is 20 mL.

Still further, the first reaction is conducted at 23° C. to 65° C. with a residence time of 100 s to 200 s; the second reaction is conducted at −35° C. to −10° C. with a residence time of 80 s to 180 s; and the third reaction is conducted at 12° C. to 50° C. with a residence time of 30 s to 55 s.

Preferably, the first reaction is conducted at 25° C. to 60° C. with a residence time of 120 s to 180 s; the second reaction is conducted at −30° C. to −20° C. with a residence time of 100 s to 150 s; and the third reaction is conducted at 15° C. to 45° C. with a residence time of 35 s to 50 s.

Still further, the brominated benzo-fused nitrogen heterocycle, the dehydrogenation reagent, the debromination reagent, and the amide compound are in a molar ratio of 1:1:2:2-4.

Still further, solvents in the solution including the brominated benzo-fused nitrogen heterocycle, the solution including the dehydrogenation reagent, the solution including the debromination reagent, and the solution including the amide compound are each anhydrous tetrahydrofuran or n-hexane;
a concentration of the solution including the brominated benzo-fused nitrogen heterocycle is 0.8 mol/L to 1.5 mol/L, and a flow rate of the solution including the brominated benzo-fused nitrogen heterocycle in a reactor is 10 mL/min to 30 mL/min;
a concentration of the solution including the dehydrogenation reagent is 0.8 mol/L to 2 mol/L, and a flow rate of the solution including the dehydrogenation reagent in a reactor is 20 mL/min to 40 mL/min;
a concentration of the solution including the debromination reagent is 0.8 mol/L to 2 mol/L, and a flow rate of the solution including the debromination reagent in a reactor is 20 mL/min to 40 mL/min; and
a concentration of the solution including the amide compound is 1.5 mol/L to 5 mol/L, and a flow rate of the solution including the amide compound in a reactor is 10 mL/min to 30 mL/min.

Still further, the post-treatment includes: cooling a third material produced from the third continuous flow microchannel reactor to below 5° C., adding a phosphoric acid aqueous solution to quench a reaction, and conducting extraction with an ester solvent; and combining resulting organic phases, washing, drying, removing the solvent, and recrystallizing.

### Beneficial Technical Effects

The present disclosure develops a method for introducing acyl on a benzene ring of a brominated benzo-fused nitrogen heterocycle. This method achieves the efficient synthesis of an acyl-substituted benzo-fused nitrogen heterocycle based on a continuous flow microchannel technology. In this method, an organic alkali (such as sodium bis(trimethylsilyl)amide (HMDSNa)) is used as a dehydrogenation reagent to remove active hydrogen from an N atom in a nitrogen heterocyclic ring of the brominated benzo-fused nitrogen heterocycle, then alkyllithium is used as a debromination reagent to remove bromine from a benzene ring, and then a reaction with an amide compound is carried out in a microchannel. As a result, this method enables mild reaction conditions, and does not require the temperature of −78° C. in the conventional technique. An intermediate generated during a reaction does not need to be separated and can be directly delivered to the next reaction. After the final reaction is completed, only simple quenching and extraction are required to produce a high-purity acyl-substituted benzo-fused nitrogen heterocycle.

The reaction of the present disclosure involves relatively-mild conditions, simple raw materials, a high yield, and a high purity, enables the continuous large-scale production, and leads to minimal “three wastes” (waste gas, waste water, and waste residues). Therefore, the method of the present disclosure is eco-friendly, and is suitable for large-scale industrial production.

### DESCRIPTION OF THE DRAWINGS

FIG. 1 is a schematic diagram of a process for preparing indole-5-carbaldehyde based on a continuous flow microchannel technology.

### SPECIFIC IMPLEMENTATIONS

The technical solutions in the embodiments of the present disclosure will be clearly and completely described below with reference to the embodiments and the accompanying drawings of the present disclosure. Obviously, the described embodiments are merely some rather than all of the embodiments of the present disclosure. The following description of at least one exemplary embodiment is merely illustrative, and is not intended to limit the present disclosure and application or use thereof in any way. All other embodiments obtained by a person of ordinary skill in the art based on the embodiments of the present disclosure without creative efforts shall fall within the protection scope of the present disclosure.

Unless otherwise specified, the numerical values set forth in these embodiments do not limit the scope of the present disclosure. The techniques and methods known to those of ordinary skill in the relevant arts may not be discussed in detail, but where appropriate, the techniques and methods should be regarded as a part of the description. In all examples shown and discussed herein, any specific value should be interpreted as merely exemplary, rather than restrictive. Therefore, other examples of the exemplary embodiments may have different values.

In addition, it should be noted that the terms such as “first” and “second” are used to specify a reaction temperature, a continuous flow microchannel reactor, etc., and are merely provided for the convenience of distinguishing between reaction steps. Unless otherwise stated, these terms do not have special meanings and should not be understood as limiting the protection scope of the present disclosure.

In the following embodiments, any experimental methods which are not specified with specific conditions are generally implemented according to the national standards, and are implemented according to the general standard requirements or methods if there are no corresponding national standards.

The yield below refers to a mass product expressed in a unit of wt %, and the “wt” will be omitted below.

The residence time refers to a time required for a material to pass through a continuous flow microchannel reactor, and is specifically calculated as follows: residence time=liquid-holding volume×(60 s/min)/total volume flow rate,
where the liquid-holding volume is a capacity of a continuous flow microchannel reactor, and a liquid-holding volume of a single glass chip is a fixed value of 20 mL; and
the total volume flow rate is a sum of flow rates of all syringe pumps.

### Example 1

### Preparation of indole-5-carbaldehyde by the conventional method

5-bromoindole (19.6 g, 0.1 mol) was added to a 500 mL three-neck flask, 100 mL of anhydrous tetrahydrofuran was added under nitrogen protection, and stirring was conducted until the 5-bromoindole was completely dissolved. Sodium bis(trimethylsilyl)amide (100 mL, 1 M, tetrahydrofuran solution) was added, and stirring was further conducted at room temperature for 1 h. A resulting reaction solution was cooled to −78° C., a n-butyllithium solution (125 mL, 1.6 M, n-hexane solution) was slowly added dropwise, and after the dropwise addition was completed, stirring was further conducted for 1 h. DMF (0.2 mol, 15.5 mL was dissolved in 85 mL of tetrahydrofuran) was finally added, a reaction temperature was raised to room temperature, and stirring was further conducted. It was monitored by thin layer chromatography (TLC) until a reaction was completed. A resulting reaction system was cooled to 0° C., and a 1 M H₃PO₄ aqueous solution was slowly added to quench the reaction. Extraction was conducted with ethyl acetate. Resulting organic phases were combined, washed with a saturated NaHCO₃ aqueous solution, dried with anhydrous MgSO₄, and subjected to rotary evaporation to produce a crude product. Recrystallization was conducted with ethanol to produce the indole-5-carbaldehyde, with a yield of 89 wt %.

The product of this example was subjected to proton and carbon nuclear magnetic resonance spectroscopy, and results were as follows:

¹H NMR (400 MHz, CDCl₃) δ 10.04 (s, 1H), 8.53 (s, 1H), 8.19 (s, 1H), 7.79 (dd, J=8.5, 1.5 Hz, 1H), 7.49 (d, J=8.5 Hz, 1H), 7.35-7.30 (m, 1H), 6.74-6.69 (m, 1H); and

¹³C NMR (101 MHz, CDCl₃) δ 192.74, 139.37, 129.78, 127.74, 126.38, 126.10, 122.31, 111.76, 104.50.

### Example 2

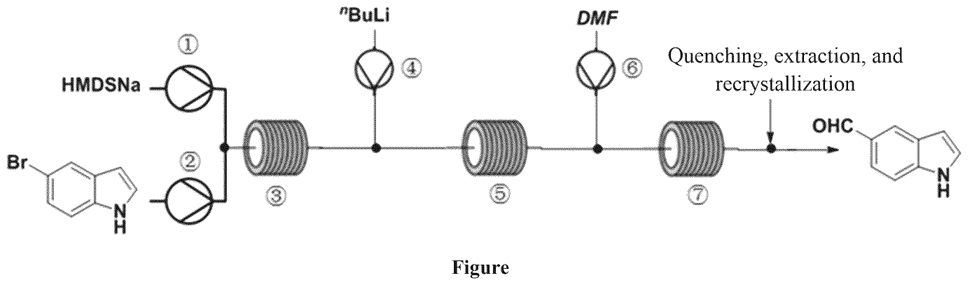

A method for aryl debromination and acylation of a brominated benzo-fused nitrogen heterocycle was provided, which was specifically the preparation of indole-5-carbaldehyde based on a continuous flow microchannel technique and involved a process flow shown in FIG. 1. The method included the following steps:

S1: Preparation of a 5-bromoindole solution: 5-bromoindole (raw material, 19.6 g, 0.1 mol) was added to a 500 mL three-neck flask, 80 mL of anhydrous tetrahydrofuran was added under nitrogen protection, and stirring was conducted until the 5-bromoindole was completely dissolved.

A sodium bis(trimethylsilyl)amide solution (alkali, HMDSNa, 100 mL, 1 M tetrahydrofuran solution) and the 5-bromoindole solution (100 mL, 1 M tetrahydrofuran solution) were introduced into first continuous flow microchannel reactor (3) through syringe pump (1) and syringe pump (2), respectively, with a flow rate set to 10 mL/min (or the sodium bis(trimethylsilyl)amide solution was added to the solution of 5-bromoindole in tetrahydrofuran, mixing was conducted thoroughly, and a resulting mixed solution was introduced into the first continuous flow microchannel reactor (3) through a syringe pump, with a flow rate set to 20 mL/min). A mixed material underwent a first reaction in the first continuous flow microchannel reactor (3). A temperature in the first continuous flow microchannel reactor (3) was set to 25° C., and a residence time of the mixed material in the first continuous flow microchannel reactor was 3 min. An effluent from the first continuous flow microchannel reactor (3) was a first material.

S2: Preparation of a diluted i-butyllithium solution: A i-butyllithium solution (nBuLi, 125 mL, 1.6 M n-hexane solution) was cooled to −20° C., and 75 mL of an anhydrous tetrahydrofuran solution was added to produce a 1 M diluted n-butyllithium solution.

The first material and the diluted n-butyllithium solution injected through syringe pump (4) were simultaneously introduced into second continuous flow microchannel reactor (5) to produce a mixed material, and the mixed material underwent a second reaction. A flow rate of the diluted n-butyllithium solution was set to 20 mL/min, a temperature in the second continuous flow microchannel reactor (5) was set to −20° C., and a residence time of the mixed material in the second continuous flow microchannel reactor (5) was 2.5 min. An effluent from the second continuous flow microchannel reactor (5) was a second material.

S3: Preparation of a DMF solution: Anhydrous DMF (nucleophile, 15.5 mL, 0.2 mol) was dissolved in 85 mL of an anhydrous tetrahydrofuran solution, and mixing was conducted thoroughly to produce a solution of 2 M DMF in tetrahydrofuran.

The second material and the DMF solution injected through syringe pump (6) were simultaneously introduced into second continuous flow microchannel reactor (7) to produce a mixed material, and the mixed material underwent a third reaction. A flow rate of the DMF solution was set to 10 mL/min, a temperature in the third continuous flow microchannel reactor (7) was set to 25° C., and a residence time of the mixed material in the third continuous flow microchannel reactor (7) was 48 s. An effluent from the third continuous flow microchannel reactor (7) was collected to produce a third material.

Post-treatment: The third material was cooled to 0° C., and a 1 M H₃PO₄ aqueous solution was slowly added to quench a reaction. Extraction was conducted with ethyl acetate. Resulting organic phases were combined, washed with a saturated NaHCO₃ aqueous solution, dried with anhydrous MgSO₄, and subjected to vacuum distillation for removing the solvent to produce a crude product. Recrystallization was conducted with ethanol to produce refined indole-5-carbaldehyde.

A yield of the product in this example was shown in Table 1. Peak positions of proton and carbon nuclear magnetic resonance spectra for the product were the same as those in Example 1.

### Example 3

In this example, indole-5-carbaldehyde was prepared based on a continuous flow microchannel technique. A specific preparation process was the same as the preparation process in Example 2, except that the temperature in the first continuous flow microchannel reactor (3) was set to 40° C.

A yield of the product in this example was shown in Table 1. Peak positions of proton and carbon nuclear magnetic resonance spectra for the product were the same as those in Example 1.

### Example 4

In this example, indole-5-carbaldehyde was prepared based on a continuous flow microchannel technique. A specific preparation process was the same as the preparation process in Example 2, except that the temperature in the first continuous flow microchannel reactor (3) was set to 60° C.

A yield of the product in this example was shown in Table 1. Peak positions of proton and carbon nuclear magnetic resonance spectra for the product were the same as those in Example 1.

### Example 5

In this example, indole-5-carbaldehyde was prepared based on a continuous flow microchannel technique. A specific preparation process was the same as the preparation process in Example 2, except that the temperature in the second continuous flow microchannel reactor (5) was set to −30° C.

A yield of the product in this example was shown in Table 1. Peak positions of proton and carbon nuclear magnetic resonance spectra for the product were the same as those in Example 1.

### Example 6

In this example, indole-5-carbaldehyde was prepared based on a continuous flow microchannel technique. A specific preparation process was the same as the preparation process in Example 2, except that the temperature in the second continuous flow microchannel reactor (5) was set to −10° C.

A yield of the product in this example was shown in Table 1. Peak positions of proton and carbon nuclear magnetic resonance spectra for the product were the same as those in Example 1.

### Example 7

In this example, indole-5-carbaldehyde was prepared based on a continuous flow microchannel technique. A specific preparation process was the same as the preparation process in Example 2, except that the temperature in the second continuous flow microchannel reactor (5) was set to 0° C.

A yield of the product in this example was shown in Table 1. Peak positions of proton and carbon nuclear magnetic resonance spectra for the product were the same as those in Example 1.

### Example 8

In this example, indole-5-carbaldehyde was prepared based on a continuous flow microchannel technique. A specific preparation process was the same as the preparation process in Example 2, except that the temperature in the third continuous flow microchannel reactor (7) was set to 15° C.

A yield of the product in this example was shown in Table 1. Peak positions of proton and carbon nuclear magnetic resonance spectra for the product were the same as those in Example 1.

### Example 9

In this example, indole-5-carbaldehyde was prepared based on a continuous flow microchannel technique. A specific preparation process was the same as the preparation process in Example 2, except that the temperature in the third continuous flow microchannel reactor (7) was set to 35° C.

A yield of the product in this example was shown in Table 1. Peak positions of proton and carbon nuclear magnetic resonance spectra for the product were the same as those in Example 1.

### Example 10

In this example, indole-5-carbaldehyde was prepared based on a continuous flow microchannel technique. A specific preparation process was the same as the preparation process in Example 2, except that the temperature in the third continuous flow microchannel reactor (7) was set to 45° C.

A yield of the product in this example was shown in Table 1. Peak positions of proton and carbon nuclear magnetic resonance spectra for the product were the same as those in Example 1.

### Example 11

In this example, indole-5-carbaldehyde was prepared based on a continuous flow microchannel technique. A specific preparation process was the same as the preparation process in Example 2, except that, in the S2, the residence time of the mixed material in the second continuous flow microchannel reactor (5) was 2 min and the flow rate of the diluted n-butyllithium solution was set to 30 mIUmin; and in the S3, the residence time of the mixed material in the third continuous flow microchannel reactor (7) was 40 s.

A yield of the product in this example was shown in Table 1. Peak positions of proton and carbon nuclear magnetic resonance spectra for the product were the same as those in Example 1.

### Example 12

In this example, indole-5-carbaldehyde was prepared based on a continuous flow microchannel technique. A specific preparation process was the same as the preparation process in Example 2, except that, in the S2, the residence time of the mixed material in the second continuous flow microchannel reactor (5) was 100 s and the flow rate of the diluted n-butyllithium solution was set to 40 mL/min; and in the S3, the residence time of the mixed material in the third continuous flow microchannel reactor (7) was 35 s.

A yield of the product in this example was shown in Table 1. Peak positions of proton and carbon nuclear magnetic resonance spectra for the product were the same as those in Example 1.

### Example 13

In this example, indole-5-carbaldehyde was prepared based on a continuous flow microchannel technique. A specific preparation process was the same as the preparation process in Example 2, except that, in the S1, the residence time of the mixed material (if there were two separate materials, flow rates of the two separate materials were set to 20 mUmin and 10 mL/min, respectively; and if there was a mixed material, a flow rate of the mixed material was set to 30 mL/min) in the first continuous flow microchannel reactor (3) was 120 s; in the S2, the residence time of the mixed material in the second continuous flow microchannel reactor (5) was 120 s; and in the S3, the residence time of the mixed material in the third continuous flow microchannel reactor (7) was 40 s.

A yield of the product in this example was shown in Table 1. Peak positions of proton and carbon nuclear magnetic resonance spectra for the product were the same as those in Example 1.

### Example 14

In this example, indole-5-carbaldehyde was prepared based on a continuous flow microchannel technique. A specific preparation process was the same as the preparation process in Example 2, except that: In the S3, preparation of a DMF solution: anhydrous DMF (8 mL, 0.1 mol) was dissolved in 92 mL of an anhydrous tetrahydrofuran solution, and mixing was conducted thoroughly to produce a solution of 1 M DMF in tetrahydrofuran. A flow rate of the solution of DMF in tetrahydrofuran was set to 20 mL/min. In the S3, the residence time of the mixed material in the third continuous flow microchannel reactor (7) was 40 s.

That is, in Examples 2 to 13, n_{(raw material)}:n₍ₐₗₖₐₗᵢ₎:n_{(nBuLi)}:n_{(nucleophile)}=1:1:2:2, but in this example, n_{(raw material)}:n₍ₐₗₖₐₗᵢ₎:n_{(nBuLi)}:n_{(nucleophile)}=1:1:2:1.

A yield of the product in this example was shown in Table 1. Peak positions of proton and carbon nuclear magnetic resonance spectra for the product were the same as those in Example 1.

### Example 15

In this example, indole-5-carbaldehyde was prepared based on a continuous flow microchannel technique. A specific preparation process was the same as the preparation process in Example 2, except that, in the S3, preparation of a DMF solution: anhydrous DMF (31 mL, 0.4 mol) was dissolved in 70 mL of an anhydrous tetrahydrofuran solution, and mixing was conducted thoroughly to produce a solution of 1 M DMF in tetrahydrofuran.

That is, in Examples 2 to 13, n_{(raw material)}:n₍ₐₗₖₐₗᵢ₎:n_{(nBuLi)}:n_{(nucleophile)}=1:1:2:2, but in this example, n_{(raw material)}:n₍ₐₗₖₐₗᵢ₎:n_{(nBuLi)}:n_{(nucleophile)}=1:1:2:4.

A yield of the product in this example was shown in Table 1. Peak positions of proton and carbon nuclear magnetic resonance spectra for the product were the same as those in Example 1.

The reaction conditions and yields for the product indole-5-carbaldehyde in Examples 1 to 15 were shown in Table 1.

TABLE 1
Yields for the product indole-5-carbaldehyde under different reaction conditions in Examples 1 to 15

| | Temperatures of three | | Residence times in | |
|---|---|---|---|---|
| | microchannel reactors | | three microchannel | |
| Example | (° C.) | n_{(raw material)}:n₍ₐₗₖₐₗᵢ₎:n_{(nBuLi)}:n_{(nucleophile)} | (s) | Yield |
| 1 | Traditional reaction | 1:1:2:2 | Traditional reaction | 89% |
| | Room | | 1 h/1 h/TLC | |
| | temperature/−78° C./room | | | |
| | temperature | | | |
| 2 | 25/−20/25 | 1:1:2:2 | 180/150/48 | 93% |
| 3 | 40/−20/25 | 1:1:2:2 | 180/150/48 | 94% |
| 4 | 60/−20/25 | 1:1:2:2 | 180/150/48 | 93% |
| 5 | 25/−30/25 | 1:1:2:2 | 180/150/48 | 96% |
| 6 | 25/−10/25 | 1:1:2:2 | 180/150/48 | 69% |
| 7 | 25/0/25 | 1:1:2:2 | 180/150/48 | 28% |
| 8 | 25/−20/15 | 1:1:2:2 | 180/150/48 | 90% |
| 9 | 25/−20/35 | 1:1:2:2 | 180/150/48 | 94% |
| 10 | 25/−20/45 | 1:1:2:2 | 180/150/48 | 94% |
| 11 | 25/−20/25 | 1:1:2:2 | 180/120/40 | 90% |
| 12 | 25/−20/25 | 1:1:2:2 | 180/100/35 | 92% |
| 13 | 25/−20/25 | 1:1:2:2 | 120/120/40 | 97% |
| 14 | 25/−20/25 | 1:1:2:1 | 180/150/40 | 79% |
| 15 | 25/−20/25 | 1:1:2:4 | 180/150/48 | 99% |

As shown in Table 1, when the brominated benzo-fused nitrogen heterocycle (raw material), the dehydrogenation reagent (alkali), the debromination reagent (nBuLi), and the amide compound (nucleophile) are in a molar ratio of 1:1:2:2-4, the first reaction in the first continuous flow microchannel reactor is conducted at preferably 25° C. to 60° C. with a residence time of preferably 120 s to 180 s, the second reaction in the second continuous flow microchannel reactor is conducted at preferably −30° C. to −20° C. with a residence time of preferably 100 s to 150 s, and the third reaction in the third continuous flow microchannel reactor is conducted at preferably 15° C. to 45° C. with a residence time of preferably 35 s to 50 s. In this case, the yields of acyl-substituted benzo-fused nitrogen heterocycles (the product indole-5-carbaldehyde in Examples 2 to 15) are 90% or more, and even can reach 99%.

### Example 16

In this example, carbazole-3-carbaldehyde was prepared based on a continuous flow microchannel technique. A specific preparation process was the same as the preparation process in Example 15, except that: the raw material was 3-bromocarbazole (amount: 24.4 g, 0.1 mol), the nucleophile was DMF (amount: 31 mL, 0.4 mol), the product was carbazole-3-carbaldehyde, and the yield was 97%.

The product of this example was subjected to proton and carbon nuclear magnetic resonance spectroscopy, and results were as follows: ¹H NMR (400 MHz, CDCl₃): δ 10.10 (s, 1H), 8.61 (s, 1H), 8.56 (s, 1H), 8.13 (d, J=8.0 Hz, 1H), 7.98 (dd, J=8.4, 1.6 Hz, 1H), 7.52-7.47 (m, 3H), 7.36-7.26 (m, 1H);

¹³C NMR (100 MHz, CDCl₃): δ 192.03, 143.33, 140.00, 129.07, 127.34, 126.97, 124.11, 123.56, 123.22, 120.76, 120.73, 111.17, 110.97.

### Example 17

In this example, 8-methoxy-9H-carbazole-3-carbaldehyde was prepared based on a continuous flow microchannel technique. A specific preparation process was the same as the preparation process in Example 15, except that: the raw material was 8-methoxy-3-bromocarbazole (CAS No.: 1916473-46-5, amount: 27.5 g, 0.1 mol), the nucleophile was DMF (amount: 31 mL, 0.4 mol), the product was 8-methoxy-9H-carbazole-3-carbaldehyde, and the yield was 87%.

The product of this example was subjected to proton and carbon nuclear magnetic resonance spectroscopy, and results were as follows: 1H NMR (500 MHz, CDCl₃): δ=4.03 (s, 3H), 6.97 (d, J=7.9 Hz, 1H), 7.25 (t, J=7.9 Hz, 1H), 7.53 (d, J=8.5 Hz, 1H), 7.73 (d, J=7.9 Hz, 1H), 7.98 (dd, J=8.5, 1.6 Hz, 1H), 8.59 (d, J=0.6 Hz, 1H), 8.62 (br. s, 1H), 10.10 (s, 1H) ppm.;

¹³C NMR and DEPT (125 MHz, CDCl₃): δ=55.75, 107.07, 111.40, 113.14, 121.35, 123.97, 124.37, 124.70, 127.17, 129.22, 130.42, 142.95, 145.97, 192.06.

### Example 18

In this example, 1-methyl-9H-carbazole-3-carbaldehyde was prepared based on a continuous flow microchannel technique. A specific preparation process was the same as the preparation process in Example 15, except that: the raw material was 1-methyl-3-bromo-carbazole (amount: 25.8 g, 0.1 mol, the raw material was synthesized by the method in CN113480470A: with the corresponding mono-substituted diphenylamine as a starting material, ring-closing was conducted first and then bromination was conducted, or the corresponding mono-substituted carbazole was brominated directly at the position 3 with N-bromosuccinimide (NBS), Discovery and characterization of a potent and selective antagonist of melanin-concentrating hormone receptor 2 [J], Bioorganic & Medicinal Chemistry Letters, 2012, 22 (1), P363-366), the nucleophile was DMF (amount: 31 mL, 0.4 mol), the product was 1-methyl-9H-carbazole-3-carbaldehyde, and the yield was 91%.

The product of this example was subjected to proton and carbon nuclear magnetic resonance spectroscopy, and results were as follows: ¹H NMR (500 MHz, CDCl₃): δ=2.62 (s, 3H, CH₃), 7.32 (m, 1H), 7.48 (br, 1H), 7.51 (br, 1H), 7.80 (br s, 1H), 8.12 (br, 1H), 8.35 (br s, 1H), 8.45 (d, J=1.0 Hz, 1H), 10.07 (s, 1H);

¹³C NMR (125 MHz, CDCl₃): δ=16.8, 111.2, 120.4, 120.78, 120.80, 122.5, 123.0, 123.8, 126.8, 127.0, 129.4, 139.9, 142.9, 192.1.

### Example 19

In this example, 1,7-dimethyl-9H-carbazole-3-carbaldehyde was prepared based on a continuous flow microchannel technique. A specific preparation process was the same as the preparation process in Example 15, except that: the raw material was 1,7-dimethyl-3-bromo-carbazole (amount: 27.3 g, 0.1 mol, the raw material was synthesized by the method in CNI13480470A: with the corresponding disubstituted diphenylamine as a starting material, ring-closing was conducted first and then bromination was conducted, or the corresponding disubstituted carbazole was brominated directly at the position 3 with NBS, Discovery and characterization of a potent and selective antagonist of melanin-concentrating hormone receptor 2 [J], Bioorganic & Medicinal Chemistry Letters, 2012, 22 (1), P363-366), the nucleophile was DMF (amount: 31 mL, 0.4 mol), the product was 1,7-dimethyl-9H-carbazole-3-carbaldehyde, and the yield was 97%.

The product of this example was subjected to proton and carbon nuclear magnetic resonance spectroscopy, and results were as follows: ¹H NMR (500 MHz, CDCl₃): δ=2.56 (s, 3H), 2.61 (s, 3H), 7.14 (br d, J=8.0 Hz, 1H), 7.30 (br s, 1H), 7.77 (br s, 1H), 8.00 (d, J=7.9 Hz, 1H), 8.26 (br s, 1H, NH), 8.41 (br s, 1H), 10.1 (s, 1H);

¹³C NMR (125 MHz, CDCl₃): δ=16.7, 22.1, 111.3, 120.2, 120.4, 121.4, 122.1, 122.3, 123.1, 126.7, 129.3, 137.1, 140.3, 142.8, 192.1.

### Example 20

In this example, 1,6-dimethyl-9H-carbazole-3-carbaldehyde was prepared based on a continuous flow microchannel technique. A specific preparation process was the same as the preparation process in Example 15, except that: the raw material was 1,6-dimethyl-3-bromo-carbazole (amount: 27.3 g, 0.1 mol, the raw material was synthesized by the method in CN113480470A: with the corresponding disubstituted diphenylamine as a starting material, ring-closing was conducted first and then bromination was conducted, or the corresponding disubstituted carbazole was brominated directly at the position 3 with NBS, Discovery and characterization of a potent and selective antagonist of melanin-concentrating hormone receptor 2 [J], Bioorganic & Medicinal Chemistry Letters, 2012, 22 (1), P363-366), the nucleophile was DMF (amount: 31 mL, 0.4 mol), the product was 1,6-dimethyl-9H-carbazole-3-carbaldehyde, and the yield was 91%.

The product of this example was subjected to proton and carbon nuclear magnetic resonance spectroscopy, and results were as follows: ¹H NMR (500 MHz, CDCl₃): δ=2.56 (s, 3H), 2.61 (s, 3H), 7.30 (m, 1H), 7.40 (d, J=7.8 Hz, 1H), 7.78 (br s, 1H), 7.91 (br s, 1H), 8.29 (br s, 1H, NH), 8.42 (br s, 1H), 10.07 (s, 1H);

¹³C NMR (125 MHz, CDCl₃): δ=16.7, 21.5, 110.9, 120.3, 120.7, 122.6, 122.8, 123.9, 126.8, 128.1, 129.2, 130.3, 138.0, 143.1, 192.1.

### Example 21

In this example, 7-methoxy-9H-carbazole-3-carbaldehyde was prepared based on a continuous flow microchannel technique. A specific preparation process was the same as the preparation process in Example 15, except that: the raw material was 7-methoxy-3-bromo-carbazole (CAS No.: 1353492-63-3, amount: 28.9 g, 0.1 mol), the nucleophile was DMF (amount: 31 mL, 0.4 mol), the product was 7-methoxy-9H-carbazole-3-carbaldehyde, and the yield was 93%.

The product of this example was subjected to proton and carbon nuclear magnetic resonance spectroscopy, and results were as follows: ¹H NMR (500 MHz, acetone-d6): δ=2.62 (s, 3H), 3.88 (s, 3H), 6.90 (m, 1H), 7.08 (d, J=2.2 Hz, 1H), 7.69 (br s, 1H), 8.09 (d, J=8.5 Hz, 1H), 8.42 (br s, 1H), 10.03 (s, 1H), 10.65 (br, 1H, N—H);

¹³C NMR (125 MHz, acetone-d6): δ=17.6, 56.5, 96.8, 110.7, 118.7, 122.1, 122.5, 122.9, 124.6, 126.9, 131.1, 143.8, 144.8, 161.3, 192.8.

### Example 22

In this example, 6-methoxy-9H-carbazole-3-carbaldehyde was prepared based on a continuous flow microchannel technique. A specific preparation process was the same as the preparation process in Example 15, except that: the raw material was 3-bromo-6-methoxy-9H-carbazole (CAS No.: 200289-73-2, amount: 28.9 g, 0.1 mol), the nucleophile was DMF (amount: 31 mL, 0.4 mol), the product was 6-methoxy-9H-carbazole-3-carbaldehyde, and the yield was 87%.

The product of this example was subjected to proton and carbon nuclear magnetic resonance spectroscopy, and results were as follows: ¹H NMR (500 MHz, CDCl₃/DMSO-d₆, 9:1): δ=2.55 (s, 3H), 3.85 (s, 3H), 7.00 (m, 1H), 7.39 (d, J=8.5 Hz, 1H), 7.49 (d, J=2.5 Hz, 1H), 7.64 (br s, 1H), 8.31 (br s, 1H), 9.94 (s, 1H), 10.78 (br s, 1H, NH);

¹³C NMR (125 MHz, CDCl₃/DMSO-d₆ 9:1): δ=16.4, 55.2, 102.2, 111.8, 115.0, 120.4, 121.8, 122.0, 123.2, 125.4, 127.5, 134.8, 143.4, 153.5, 191.3.

### Example 23

In this example, 6,7-dimethoxy-9H-carbazole-3-carbaldehyde was prepared based on a continuous flow microchannel technique. A specific preparation process was the same as the preparation process in Example 15, except that: the raw material was 6,7-dimethoxy-3-bromo-carbazole (amount: 32.0 g, 0.1 mol), the nucleophile was DMF (amount: 31 mL, 0.4 mol), the product was 6,7-dimethoxy-9H-carbazole-3-carbaldehyde, and the yield was 83%.

The product of this example was subjected to proton and carbon nuclear magnetic resonance spectroscopy, and results were as follows: ¹H NMR (500 MHz, CDCl₃): δ=2.59 (s, 3H), 3.98 (s, 3H), 4.02 (s, 3H), 7.02 (s, 1H), 7.55 (s, 1H), 7.71 (br s, 1H), 8.23 (br s, 1H, NH), 8.34 (br s, 1H), 10.06 (s, 1H);

¹³C NMR (125 MHz, CDCl₃): δ=16.7, 56.2, 56.5, 94.7, 102.7, 115.9, 120.1, 121.4, 123.3, 125.7, 129.2, 134.5, 142.6, 145.4, 150.0, 192.2.

### Example 24

In this example, indole-4-carbaldehyde was prepared based on a continuous flow microchannel technique. A specific preparation process was the same as the preparation process in Example 15, except that: the raw material was 4-bromoindole (amount: 19.6 g, 0.1 mol), the nucleophile was DMF (amount: 31 mL, 0.4 mol), the product was indole-4-carbaldehyde, and the yield was 91%.

The product of this example was subjected to proton nuclear magnetic resonance spectroscopy, and results were as follows: ¹HNMR (400 MHz, CDCl₃): δ=7.3-7.45 (m, 3H), 7.6-7.7 (m, 2H), 8.65 (br s, 1H), 10.4 (s, 1H).

### Example 25

In this example, indole-6-carbaldehyde was prepared based on a continuous flow microchannel technique. A specific preparation process was the same as the preparation process in Example 15, except that: the raw material was 6-bromoindole (amount: 19.6 g, 0.1 mol), the nucleophile was DMF (amount: 31 mL, 0.4 mol), the product was indole-6-carbaldehyde, and the yield was 95%.

The product of this example was subjected to proton nuclear magnetic resonance spectroscopy, and results were as follows: ¹NMR (400 MHz, CDCl₃): δ=6.64 (br s, 1H), 7.46 (m, 1H), 7.65 (d, 1H, J=8.1), 7.74 (d, 1H, J=8.1), 7.96 (s, 1H), 8.8 (br s, 1), 10.0 (s, 1H).

### Example 26

In this example, indole-7-carbaldehyde was prepared based on a continuous flow microchannel technique. A specific preparation process was the same as the preparation process in Example 15, except that: the raw material was 7-bromoindole (amount: 19.6 g, 0.1 mol), the nucleophile was DMF (amount: 31 mL, 0.4 mol), the product was indole-7-carbaldehyde, and the yield was 93%.

The product of this example was subjected to proton nuclear magnetic resonance spectroscopy, and results were as follows: ¹H NMR (400 MHz, CDCl₃): δ=6.61 (m, 1H), 7.24 (t, J=7.5, 1H), 7.32 (m, 1H), 7.62 (d, J=7.2, 1H), 7.92 (d, J=7.9, 1H).

### Example 27

In this example, 6-fluoro-indole-5-carbaldehyde was prepared based on a continuous flow microchannel technique. A specific preparation process was the same as the preparation process in Example 15, except that: the raw material was 6-fluoro-5-bromoindole (CAS No. 434960-42-6, amount: 21.2 g, 0.1 mol), the nucleophile was DMF (amount: 31 mL, 0.4 mol), the product was 6-fluoro-indole-5-carbaldehyde, and the yield was 85%.

The product of this example was subjected to proton, carbon, and fluorine nuclear magnetic resonance spectroscopy, and results were as follows: ¹H NMR (400 MHz, DMSO-d₆): δ=11.60 (s, 1H), 10.16 (s, 1H), 8.06 (d, J=6.8 Hz, 1H,), 7.47 (t, J=3.0 Hz, 1H,), 7.28 (d, J=12.0 Hz, 1H,), 6.61 (s, 1H);

¹³C NMR (100 MHz, DMSO-d₆): δ=188.2 (d, J_{CF}=5 Hz), 160.2 (d, J_{CF}=246 Hz), 139.7 (d, J_{CF}=13 Hz), 128.8 (d, J_{CF}=3 Hz), 124.9, 123.2 (d, J_{CF}=4 Hz), 117.9 (d, J_{CF}=11 Hz), 103.7, 98.4 (d, J_{CF}=25 Hz);

¹⁹F NMR (DMSO-d₆): δ=130.24.

### Example 28

In this example, 7-fluoro-indole-6-carbaldehyde was prepared based on a continuous flow microchannel technique. A specific preparation process was the same as the preparation process in Example 15, except that: the raw material was 7-fluoro-6-bromoindole (CAS No. 936901-94-9, amount: 21.2 g, 0.1 mol), the nucleophile was DMF (amount: 31 mL, 0.4 mol), the product was 7-fluoro-indole-6-carbaldehyde, and the yield was 81%.

The product of this example was subjected to proton, carbon, and fluorine nuclear magnetic resonance spectroscopy, and results were as follows: ¹H NMR (400 MHz, DMSO-d₆): δ=10.28 (s, 1H), 7.67-7.34 (m, 2H), 7.41-7.37 (m, 1H), 6.59 (s, 1H);

¹³C NMR (100 MHz, DMSO-d₆): δ=187.3 (d, 3 J_{CF}=7 Hz), 152.7 (d, J_{CF}=259 Hz), 137.0 (d, J_{CF}=8 Hz), 131.6, 123.2 (d, J_{CF}=11 Hz), 117.9, 117.0 (d, J_{CF}=3 Hz), 116.5 (d, J_{CF}=3 Hz), 103.7 (d, J_{CF}=1 Hz);

¹⁹F NMR (DMSO-d6): δ=140.48.

### Example 29

In this example, 3-methyl-indole-7-carbaldehyde was prepared based on a continuous flow microchannel technique. A specific preparation process was the same as the preparation process in Example 15, except that: the raw material was 3-methyl-7-bromoindole (CAS No. 86915-22-2, amount: 21.0 g, 0.1 mol), the nucleophile was DMF (amount: 31 mL, 0.4 mol), the product was 3-methyl-indole-7-carbaldehyde, and the yield was 92%.

The product of this example was subjected to proton nuclear magnetic resonance spectroscopy, and results were as follows: ¹H NMR (400 MHz, CDCl₃): δ=10.12 (s, 1H), 9.85 (s, 1H), 7.89 (d, J=7.8 Hz, 1H), 7.65 (d, J=8.1 Hz, 1H), 7.25 (d, J=7.6 Hz, 1H), 7.12-7.09 (m, 1H), 2.37 (d, J=1.1 Hz, 3H).

### Example 30

In this example, 3-ethyl-indole-7-carbaldehyde was prepared based on a continuous flow microchannel technique. A specific preparation process was the same as the preparation process in Example 15, except that: the raw material was 3-ethyl-7-bromoindole (CAS No. 1360962-55-5, amount: 22.4 g, 0.1 mol), the nucleophile was DMF (amount: 31 mL, 0.4 mol), the product was 3-ethyl-indole-7-carbaldehyde, and the yield was 96%.

The product of this example was subjected to proton nuclear magnetic resonance spectroscopy, and results were as follows: ¹H NMR (400 MHz, CDCl₃): δ=10.12 (s, 1H), 9.87 (s, 1H), 7.92 (d, J=7.8 Hz, 1H), 7.65 (d, J=7.3 Hz, 1H), 7.26 (d, J=7.5 Hz, 1H), 7.14-7.10 (m, 1H), 2.83 (q, J=7.5 Hz, 2H), 1.35 (t, J=7.5 Hz, 3H).

### Example 31

In this example, 3-chloro-indole-5-carbaldehyde was prepared based on a continuous flow microchannel technique. A specific preparation process was the same as the preparation process in Example 15, except that: the raw material was 3-chloro-5-bromoindole (CAS No. 1388073-38-8, amount: 17.9 g, 0.1 mol), the nucleophile was DMF (amount: 31 mL, 0.4 mol), the product was 3-chloro-indole-5-carbaldehyde, and the yield was 86%.

The product of this example was subjected to proton and carbon nuclear magnetic resonance spectroscopy, and results were as follows: ¹H NMR (400 MHz, CDCl₃): δ=7.31 (s, 2H), 4.45 (bs, 2H);

¹³C NMR (100 MHz, CDCl₃): δ=139.4, 130.2, 120.0, 107.9.

### Example 32

In this example, 5-benzoylindole was prepared based on a continuous flow microchannel technique. A specific preparation process was the same as the preparation process in Example 15, except that: the raw material was 5-bromoindole (amount: 19.6 g, 0.1 mol), the nucleophile was N-methoxy-N-methyl-benzamide (amount: 66 g, 0.4 mol), the product was 5-benzoylindole, and the yield was 97%.

The product of this example was subjected to proton and carbon nuclear magnetic resonance spectroscopy, and results were as follows: ¹H NMR (400 MHz, CDCl₃): δ=8.85 (bs, NH, 1H), 8.14 (s, 1H), 7.86-7.77 (m, 3H), 7.61-7.55 (m, 1H), 7.52-7.42 (m, 3H), 7.31-7.29 (m, 1H), 6.69-6.60 (m, 1H);

¹³C NMR (100 MHz, CDCl₃): δ=197.7, 139.0, 138.4, 131.7, 130.0, 129.6, 128.2, 127.2, 125.9, 125.4, 124.2, 111.2, 104.2.

### Example 33

In this example, 7-benzoylindole was prepared based on a continuous flow microchannel technique. A specific preparation process was the same as the preparation process in Example 15, except that: the raw material was 7-bromoindole (amount: 19.6 g, 0.1 mol), the nucleophile was N-methoxy-N-methyl-benzamide (amount: 66 g, 0.4 mol), the product was 7-benzoylindole, and the yield was 91%.

The product of this example was subjected to proton and carbon nuclear magnetic resonance spectroscopy, and results were as follows: ¹H NMR (400 MHz, CDCl₃): δ=9.79 (s, 1H), 7.90 (d, J=7.6 Hz, 1H), 7.80 (d, J=7.2 Hz, 1H), 7.53-7.39 (m, 5H), 7.25-7.21 (m, 2H), 6.74 (d, J=3.2 Hz, 1H);

¹³C NMR (100 MHz, CDCl₃): δ=189.0, 142.4, 136.1, 131.8, 131.4, 130.2, 128.7, 127.8, 126.3, 124.8, 122.2, 120.2, 104.

### Example 34

In this example, 5-acetylindole was prepared based on a continuous flow microchannel technique. A specific preparation process was the same as the preparation process in Example 15, except that: the raw material was 5-bromoindole (amount: 19.6 g, 0.1 mol), the nucleophile was N-methoxy-N-methyl-acetamide (amount: 41.2 g, 0.4 mol), the product was 5-acetylindole, and the yield was 87%.

The product of this example was subjected to proton and carbon nuclear magnetic resonance spectroscopy, and results were as follows: ¹H NMR (400 MHz, CDCl₃): δ=8.78 (s, 1H), 8.43-8.28 (m, 1H), 7.88 (dd, J=8.6, 1.7 Hz, 1H), 7.42 (dd, J=8.6, 0.9 Hz, 1H), 7.26 (s, 1H), 6.67 (m, 1H), 2.68 (d, J=2.2 Hz, 3H);

¹³C NMR (101 MHz, CDCl₃): δ=198.9, 138.7, 130.0, 127.5, 126.0, 123.3, 122.3, 111.2, 104.3, 26.8.

### Example 35

In this example, (2S)-4-[(3-(tert-butyldimethylsilyl)-2-(ethoxyformylamino)-1-oxopropyl]-1H-indole was prepared based on a continuous flow microchannel technique. A specific preparation process was the same as the preparation process in Example 15, except that: the raw material was 4-bromoindole (amount: 19.6 g, 0.1 mol), the nucleophile was N-methyl-N-methoxy-[(2S)—O-(tert-butyldimethylsilyl)-N′-(ethoxyformyl)]propanamide (amount: 33.4 g, 0.2 mol), the product was (2S)-4-[(3-(tert-butyldimethylsilyl)-2-(ethoxyformylamino)-1-oxopropyl]-1H-indole, and the yield was 87%.

The product of this example was subjected to proton nuclear magnetic resonance spectroscopy, and results were as follows: ¹H NMR (400 MHz, CDCl₃): δ=−0.21 (s, 3H), −0.19 (s, 3H), 0.75 (s, 9H), 1.23 (t, J=7.1 Hz, 3H), 3.97 (d, J=2 Hz, 2H), 4.18 (q, J=7.1 Hz, 2H), 5.52 (m, 1H), 6.0 (d, J=7.1 Hz, 1H,), 7.2-7 (m, 3H), 7.6 (d, J=6.2 Hz, 1H), 7.7 (d, J=6.1 Hz, 1H), 8.8 (br s, 1H).

### Example 36

In this example, 1H-benzimidazole-5-carbaldehyde was prepared based on a continuous flow microchannel technique. A specific preparation process was the same as the preparation process in Example 15, except that: the raw material was 5-bromobenzimidazole (CAS No. 4887-88-1, amount: 19.5 g, 0.1 mol), the nucleophile was DMF (amount: 31 mL, 0.4 mol), the product was 1H-benzimidazole-5-carbaldehyde, and the yield was 86%.

The product of this example was subjected to proton and carbon nuclear magnetic resonance spectroscopy, and results were as follows: ¹H NMR (400 MHz, CDCl₃): δ=7.55 (d, J=8.4, 1H), 7.65 (m, 1H), 8.01 (br s, 1H), 8.24 (s, 1H), 9.85 (s, 1H,);

¹³C NMR (100 MHz, CDCl₃): δ=116.3, 120.5, 124.5, 133.1, 139.7, 142.8, 145.9, 194.0.

### Example 37

In this example, 9H-benzimidazole-4-carbaldehyde was prepared based on a continuous flow microchannel technique. A specific preparation process was the same as the preparation process in Example 15, except that: the raw material was 4-bromobenzimidazole (CAS No. 83741-35-1, amount: 19.5 g, 0.1 mol), the nucleophile was DMF (amount: 31 mL, 0.4 mol), the product was 1H-benzimidazole-4-carbaldehyde, and the yield was 80%.

The product of this example was subjected to proton and carbon nuclear magnetic resonance spectroscopy, and results were as follows: ¹H NMR (300 MHz, DMSO-d₆): δ=10.24 (s, 1H), 8.33 (s, 1H), 8.00 (dd, J=8.0, 1.0 Hz, 1H), 7.83 (dd, J=7.4, 1.0 Hz, 1H), 7.40 (dd, J=8.0, 7.4 Hz, 1H);

¹³C NMR (75 MHz, DMSO) δ 191.7, 144.2, 142.5, 127.0, 124.6, 122.2, 121.4.

### Example 38

In this example, 1H-indazole-7-carbaldehyde was prepared based on a continuous flow microchannel technique. A specific preparation process was the same as the preparation process in Example 15, except that: the raw material was 7-bromo-1H-indazole (CAS No. 53857-58-2, amount: 19.5 g, 0.1 mol), the nucleophile was DMF (amount: 31 mL, 0.4 mol), the product was 1H-indazole-7-carbaldehyde, and the yield was 83%.

The product of this example was subjected to proton and carbon nuclear magnetic resonance spectroscopy, and results were as follows: ¹H NMR (300 MHz, CDCl₃): δ=12.02 (s, 1H), 10.15 (s, 1H), 8.19 (s, 1H), 8.05 (dd, J=8.0, 0.9 Hz, 1H), 7.85 (dd, J=7.1, 0.9 Hz, 1H), 7.31 (dd, J=8.1, 7.1 Hz, 1H);

¹³C NMR (75 MHz, CDCl₃) δ 192.2, 136.7, 134.9, 133.2, 128.4, 124.4, 120.7, 120.4.

### Example 39

In this example, 1H-indazole-5-carbaldehyde was prepared based on a continuous flow microchannel technique. A specific preparation process was the same as the preparation process in Example 15, except that: the raw material was 5-bromo-1H-indazole (CAS No. 53857-57-1, amount: 19.5 g, 0.1 mol), the nucleophile was DMF (amount: 31 mL, 0.4 mol), the product was 1H-indazole-5-carbaldehyde, and the yield was 88%.

The product of this example was subjected to proton nuclear magnetic resonance spectroscopy, and results were as follows: ¹H NMR (400 MHz, CDCl₃): δ=10.54 (br s, 1H, NH), 10.07 (s, 1H), 8.32 (s, 1H), 8.27 (s, 1H), 7.99 (d, J=9.0 Hz, 1H), 7.62 (d, J=9.0 Hz, 1H).

### Example 40

In this example, 2-oxoindoline-5-carbaldehyde was prepared based on a continuous flow microchannel technique. A specific preparation process was the same as the preparation process in Example 15, except that: the raw material was 5-bromoindoline (CAS No. 20870-78-4, amount: 21.1 g, 0.1 mol), the nucleophile was DMF (amount: 31 mL, 0.4 mol), the product was 2-oxoindoline-5-carbaldehyde, and the yield was 92%.

The product of this example was subjected to proton nuclear magnetic resonance spectroscopy, and results were as follows: ¹H NMR (500 MHz, DMSO-d6): δ=7.83 (d, J=1.5 Hz, 1H), 7.69-7.65 (m, 2H), 6.85 (dd, J=17.6, 10.9 Hz, 1H), 5.97 (dd, J=17.6, 0.9 Hz, 1H), 5.38 (dd, J=10.9, 0.8 Hz, 1H), 4.37 (s, 2H).

The raw materials and products in Examples 15 to 40 were shown in Table 2.

TABLE 2
Raw materials and corresponding products and yields in Examples 15 to 40

| Raw material in an example | Product and yield thereof |
|---|---|
| 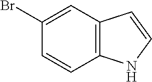 | 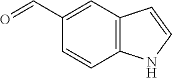 [SMILES: O=Cc1ccc2[nH]ccc2c1] |
| Example 15 | 99% |
| 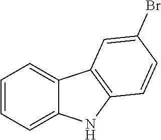 [SMILES: Brc1ccc2[nH]c3ccccc3c2c1] | 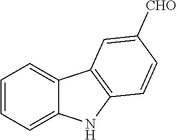 [SMILES: O=Cc1ccc2[nH]c3ccccc3c2c1] |
| Example 16 | 97% |
| 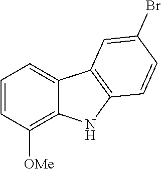 [SMILES: COc1cccc2c1[nH]c1ccc(Br)cc12] | 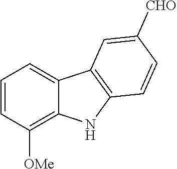 [SMILES: COc1cccc2c1[nH]c1ccc(C=O)cc12] |
| Example 17 | 87% |
| 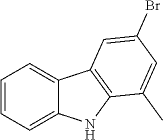 | 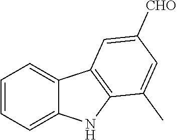 [SMILES: Cc1cc(C=O)cc2c1[nH]c1ccccc12] |
| Example 18 | 91% |
| 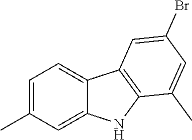 | 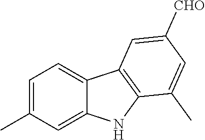 |
| Example 19 | 97% |
| 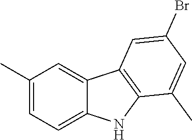 | 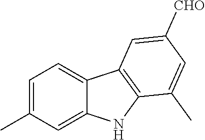 |
| Example 20 | 91% |
| 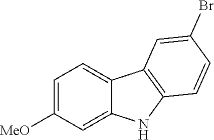 [SMILES: COc1ccc2c(c1)[nH]c1ccc(Br)cc12] | 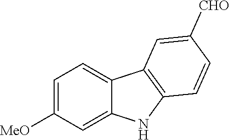 [SMILES: COc1ccc2c(c1)[nH]c1ccc(C=O)cc12] |
| Example 21 | 93% |
| 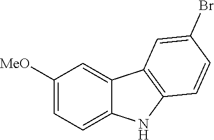 [SMILES: COc1ccc2[nH]c3ccc(Br)cc3c2c1] | 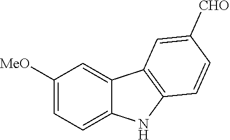 [SMILES: COc1ccc2[nH]c3ccc(C=O)cc3c2c1] |
| Example 22 | 87% |
| 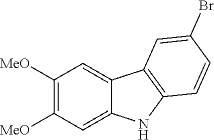 [SMILES: COc1cc2[nH]c3ccc(Br)cc3c2cc1OC] | 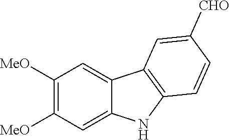 [SMILES: COc1cc2[nH]c3ccc(C=O)cc3c2cc1OC] |
| Example 23 | 83% |
| 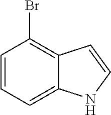 | 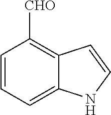 [SMILES: O=Cc1cccc2[nH]ccc12] |
| Example 24 | 91% |
| 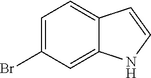 | 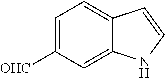 [SMILES: O=Cc1ccc2cc[nH]c2c1] |
| Example 25 | 95% |
| 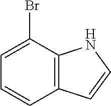 | |
| Example 26 | 93% |
| 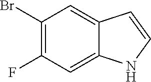 [SMILES: Fc1cc2[nH]ccc2cc1Br] | 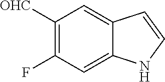 [SMILES: O=Cc1cc2cc[nH]c2cc1F] |
| Example 27 | 85% |
| 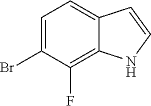 [SMILES: Fc1c(Br)ccc2cc[nH]c12] | 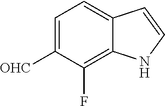 [SMILES: O=Cc1ccc2cc[nH]c2c1F] |
| Example 28 | 81% |
| 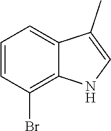 | 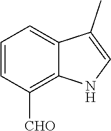 |
| Example 29 | 92% |
| 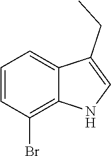 | 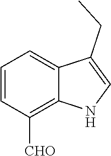 |
| Example 30 | 96% |
| 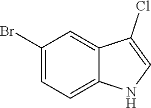 [SMILES: Clc1c[nH]c2ccc(Br)cc12] | 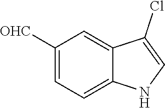 [SMILES: O=Cc1ccc2[nH]cc(Cl)c2c1] |
| Example 31 | 86% |
| 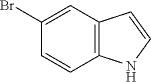 | 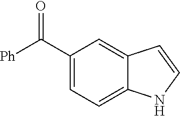 [SMILES: O=C(c1ccccc1)c1ccc2[nH]ccc2c1] |
| Example 32 | 97% |
| 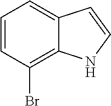 | 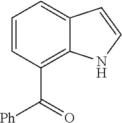 [SMILES: O=C(c1ccccc1)c1cccc2cc[nH]c12] |
| Example 33 | 91% |
| 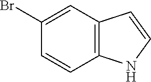 | 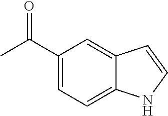 |
| Example 34 | 87% |
| 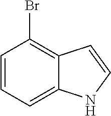 | 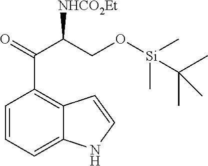 |
| Example 35 | 87% |
| 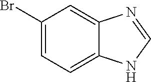 | 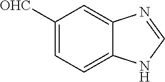 [SMILES: O=Cc1ccc2[nH]cnc2c1] |
| Example 36 | 86% |
| 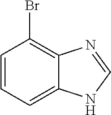 | 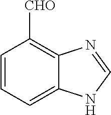 [SMILES: O=Cc1cccc2[nH]cnc12] |
| Example 37 | 80% |
| 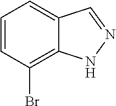 | 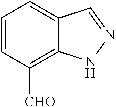 [SMILES: O=Cc1cccc2cn[nH]c12] |
| Example 38 | 83% |
| 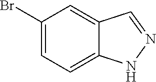 | 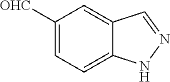 [SMILES: O=Cc1ccc2[nH]ncc2c1] |
| Example 39 | 88% |
| 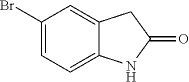 [SMILES: O=C1Cc2cc(Br)ccc2N1] | 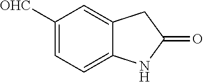 [SMILES: O=Cc1ccc2c(c1)CC(=O)N2] |
| Example 40 | 92% |

It can be seen from Table 2 that the method of the present disclosure enables the introduction of acyl on a benzene ring of a brominated benzo-fused nitrogen heterocycle, and allows a corresponding acylation product yield of 80% or more.

The above are merely preferred specific embodiments of the present disclosure, but the protection scope of the present disclosure is not limited thereto. Any equivalent replacement or modification made by a person skilled in the art according to the technical solutions of the present disclosure and the inventive concepts thereof within the technical scope of the present disclosure shall fall within the protection scope of the present disclosure.

What is claimed is:

1. A method for aryl debromination and acylation of a brominated benzo-fused nitrogen heterocycle, comprising the following steps:
S1, mixing a solution comprising the brominated benzo-fused nitrogen heterocycle with a solution comprising a dehydrogenation reagent, and conducting a first reaction to remove active hydrogen from a nitrogen heterocyclic ring to produce a first material;
S2, mixing the first material with a solution comprising a debromination reagent, and conducting a second reaction to produce a second material; and
S3, allowing the second material to undergo a third reaction with a solution comprising an amide compound, such that a nucleophilic substitution reaction occurs at a substitution position of original bromine on aryl of the brominated benzo-fused nitrogen heterocycle to produce an acylated substituent; and after the third reaction is completed, quenching the third reaction, and conducting a post-treatment to produce an acyl-substituted benzo-fused nitrogen heterocycle,
wherein the brominated benzo-fused nitrogen heterocycle is selected from one of a brominated indole, a brominated benzimidazole, a brominated indazole, a brominated indoline, and a brominated carbazole;
the dehydrogenation reagent is aminoalkylsilane; and the debromination reagent is C₁-C₂₀ n- or iso-alkyllithium; and
the first reaction is conducted in a first continuous flow microchannel reactor, the second reaction is conducted in a second continuous flow microchannel reactor, and the third reaction is conducted in a third continuous flow microchannel reactor; the first continuous flow microchannel reactor, the second continuous flow microchannel reactor, and the third continuous flow microchannel reactor communicate with each other through microchannels; and a respective raw material is injected into a reactor for each reaction through a syringe pump.

2. The method for the aryl debromination and the acylation of the brominated benzo-fused nitrogen heterocycle according to claim 1, wherein the brominated benzo-fused nitrogen heterocycle has one of the following chemical structures: 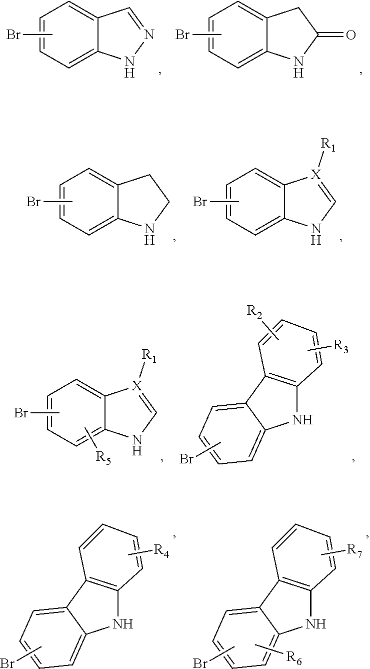
wherein X is selected from a nitrogen atom or a carbon atom, wherein when X is the nitrogen atom, R₁ is absent, and when X is the carbon atom, R₁ is selected from one of a hydrogen atom, C₁-C₆ n- or iso-alkyl, and a chlorine atom; R₅ is selected from halogen atoms;
R₂ and R₃ are each selected from one of the hydrogen atom, C₁-C₆ n- or iso-alkoxy, and the C₁-C₆ n- or iso-alkyl, and R₂ and R₃ are the same or different;
R₄ is selected from one of the hydrogen atom and the C₁-C₆ n- or iso-alkoxy;
R₆ and R₇ are each selected from one of the hydrogen atom and the C₁-C₆ n- or iso-alkyl, and R₆ and R₇ are the same or different;
the aminoalkylsilane is selected from sodium bis(trimethylsilyl)amide, potassium bis(trimethylsilyl)amide, and lithium bis(trimethylsilyl)amide;
the C₁-C₂₀ n- or iso-alkyllithium is n-butyl lithium; and
the amide compound is selected from one of N,N-dimethylformamide (DMF), N,N-dimethylacetamide, N-methoxy-N-methyl-benzamide, N-methoxy-N-methyl-acetamide, and N-methyl-N-methoxy-[(2S)—O-(tert-butyl dimethyl silyl)-N′-(ethoxyformyl)]propanamide.

3. The method for the aryl debromination and the acylation of the brominated benzo-fused nitrogen heterocycle according to claim 2, wherein the brominated benzo-fused nitrogen heterocycle is selected from one of 4-bromoindole, 5-bromoindole, 6-bromoindole, 7-bromoindole, 6-fluoro-5-bromoindole, 7-fluoro-6-bromoindole, 3-methyl-7-bromoindole, 3-ethyl-7-bromoindole, 3-chloro-7-bromoindole, 3-chloro-5-bromoindole, 3-bromocarbazole, 8-methoxy-3-bromocarbazole, 1-methyl-3-bromo-carbazole, 1,7-dimethyl-3-bromo-carbazole, 1,6-dimethyl-3-bromo-carbazole, 7-methoxy-3-bromo-carbazole, 3-bromo-6-methoxy-carbazole, 6,7-dimethoxy-3-bromo-carbazole, 4-bromobenzimidazole, 5-bromobenzimidazole, 7-bromo-indazole, 5-bromo-indazole, 5-bromo-indoline, and 5-bromo-2-oxoindoline.

4. (canceled)

5. The method for the aryl debromination and the acylation of the brominated benzo-fused nitrogen heterocycle according to claim 1, wherein the first continuous flow microchannel reactor comprises three glass chips, the second continuous flow microchannel reactor comprises five glass chips, and the third continuous flow microchannel reactor comprises two glass chips; and a liquid-holding volume of each glass chip is 20 mL.

6. The method for the aryl debromination and the acylation of the brominated benzo-fused nitrogen heterocycle according to claim 5, wherein the first reaction is conducted at 23° C. to 65° C. with a residence time of 100 s to 200 s; the second reaction is conducted at −35° C. to −10° C. with a residence time of 80 s to 180 s; and the third reaction is conducted at 12° C. to 50° C. with a residence time of 30 s to 55 s.

7. The method for the aryl debromination and the acylation of the brominated benzo-fused nitrogen heterocycle according to claim 6, wherein the first reaction is conducted at 25° C. to 60° C. with a residence time of 120 s to 180 s; the second reaction is conducted at −30° C. to −20° C. with a residence time of 100 s to 150 s; and the third reaction is conducted at 15° C. to 45° C. with a residence time of 35 s to 50 s.

8. The method for the aryl debromination and the acylation of the brominated benzo-fused nitrogen heterocycle according to claim 1, wherein the brominated benzo-fused nitrogen heterocycle, the dehydrogenation reagent, the debromination reagent, and the amide compound are in a molar ratio of 1:1:2:12-4).

9. The method for the aryl debromination and the acylation of the brominated benzo-fused nitrogen heterocycle according to claim 1, wherein solvents in the solution comprising the brominated benzo-fused nitrogen heterocycle, the solution comprising the dehydrogenation reagent, the solution comprising the debromination reagent, and the solution comprising the amide compound are each anhydrous tetrahydrofuran or n-hexane;
a concentration of the solution comprising the brominated benzo-fused nitrogen heterocycle is 0.8 mol/L to 1.5 mol/L, and a flow rate of the solution comprising the brominated benzo-fused nitrogen heterocycle in the first continuous flow microchannel reactor is 10 mL/min to 30 mL/min;
a concentration of the solution comprising the dehydrogenation reagent is 0.8 mol/L to 2 mol/L, and a flow rate of the solution comprising the dehydrogenation reagent in the first continuous flow microchannel reactor is 20 mL/min to 40 mL/min;
a concentration of the solution comprising the debromination reagent is 0.8 mol/L to 2 mol/L, and a flow rate of the solution comprising the debromination reagent in the second continuous flow microchannel reactor is 20 mL/min to 40 mL/min; and
a concentration of the solution comprising the amide compound is 1.5 mol/L to 5 mol/L, and a flow rate of the solution comprising the amide compound in the third continuous flow microchannel reactor is 10 mL/min to 30 mL/min.

10. The method for the aryl debromination and the acylation of the brominated benzo-fused nitrogen heterocycle according to claim 1, wherein the post-treatment comprises: cooling a third material produced from the third continuous flow microchannel reactor to below 5° C., adding a phosphoric acid aqueous solution to quench the third reaction, and conducting extraction with an ester solvent; and combining resulting organic phases, washing, drying, removing the ester solvent, and recrystallizing.
